(19) United States
(12) Patent Application Publication
(10) Pub. No.: US 2010/0092544 A1
(43) Pub. Date: Apr. 15, 2010

(54) Adhesive composition for patch and use thereof

(76) Inventors: Katsuhiro Okada, Ibaraki-shi (JP); Yoshihiro Iwao, Ibaraki-shi (JP); Katsuyuki Ookubo, Ibaraki-shi (JP)

Correspondence Address: WENDEROTH, LIND & PONACK, L.L.P., 1030 15th Street, N.W.,, Suite 400 East, Washington, DC, 20005-1503, US

(21) Appl. No.: 12/285,735

(22) Filed: Oct. 14, 2008

(51) Int. Cl. A61K 9/70; C09J 193/04; C09J 145/02; C09J 125/04

(52) U.S. Cl. 424/443; 525/185; 524/274; 525/186; 525/241

(57) ABSTRACT

The present invention provides an adhesive composition for patch, containing a rubber elastomer and a tackifier having a weight average molecular weight of 1200-2500, a patch having a support and an adhesive layer containing the composition, which is provided on at least one surface of the support, and a patch preparation having an adhesive layer containing a percutaneously absorbable drug (excluding bisoprolol).

### TECHNICAL FIELD OF THE INVENTION

The present invention relates to an adhesive composition for patch and use thereof.

### BACKGROUND OF THE INVENTION

In recent years, various patches and patch preparations have been developed. These patches and patch preparations are extremely superior from the aspects of wound protection and continuous transdermal administration of drugs. Such patches and patch preparations have a structure wherein an adhesive layer is laminated on a support such as a cloth, a plastic film and the like, and a release film (release liner) is laminated on the adhesive layer.

Since the adhesive layer of such patch is adhered to the skin of the body, various properties such as balance of appropriate hydrophilicity/hydrophobicity, low skin irritation and the like, which are different from those of regular industrial tapes, need to be satisfied. In some of these patches, an organic liquid component may be added to the adhesive layer to achieve good skin adhesion and decreased physical skin irritation on detachment.

For example, JP-A-06-145052 discloses a patch preparation provided with an adhesive layer comprising polyisobutylene and polybutene having a viscosity average molecular weight of 400,000-1,800,000, and describes that the adhesive layer contains an organic liquid component such as fatty acid ester and the like in a proportion of 0-15%. However, when such patch preparation contains a large amount of an organic liquid component in the adhesive layer, the adhesive layer is plasticized and has decreased cohesive force, and a part of the adhesive layer possibly remains on the skin surface on detachment of the patch or patch preparation (what is called an adhesive residue) and the like. In addition, when the cohesive force of the adhesive layer decreases, for example, a part of the adhesive remains on the release surface of a release film (adhesive residue) when the release film is removed from the patch, which in turn possibly decreases the amount of the drug contained in the preparation and decreases the treatment effect.

Furthermore, in a patch containing such an organic liquid component, the component is more easily diffused and more easily moves as the molecular weight of the organic liquid component becomes lower, due to which the organic liquid component sometimes exudes to the surface of the adhesive layer to reach the release film. When such a phenomenon occurs, the adhesive force between the adhesive layer and the release film decreases, and an adhesive residue on the release film, delamination of the release film from the adhesive layer and the like are possibly developed during production or use of the patch.

As a method to solve the above-mentioned problem of an adhesive remaining (transfer) on an adherend or a release film, which is so-called an adhesive residue, generally, a method including crosslinking an adhesive (polymer component) in an adhesive layer by adding a crosslinking agent to the adhesive layer, or a method including enhancing the cohesive force of an adhesive layer by adding a filler (JP-A-06-65066) can be used. However, the method including crosslinking a polymer component sometimes fails to achieve crosslinking due to a crosslinking inhibition by a drug or a transdermal absorption promoter to be added. On the other hand, the method including addition of a filler sometimes faces difficulty in providing a sufficient effect.

### SUMMARY OF THE INVENTION

The present invention has been made in such actual situations and aims to provide an adhesive composition for patch, which can simultaneously achieve a high cohesive force and a high level of adhesion to the skin. Moreover, the present invention aims to provide a patch and a patch preparation provided with an adhesive layer comprising the adhesive composition for patch.

The present inventors have conducted intensive studies in an attempt to solve the aforementioned problems, and found that the problems can be solved by combining a rubber elastomer constituting the adhesive composition for patch, and a tackifier having a particular molecular weight, which resulted in the completion of the present invention.

Accordingly, the present invention provides the following.

(1) An adhesive composition for patch, comprising a rubber elastomer and a tackifier having a weight average molecular weight of 1200-2500.
(2) The adhesive composition of the above-mentioned (1), wherein the above-mentioned tackifier is at least one kind selected from a petroleum resin, a terpene resin, a rosin resin, a coumarone-indene resin and a styrene resin.
(3) The adhesive composition of the above-mentioned (2), wherein the above-mentioned petroleum resin is a saturated alicyclic hydrocarbon resin having a repeat unit represented by the following formula (1):

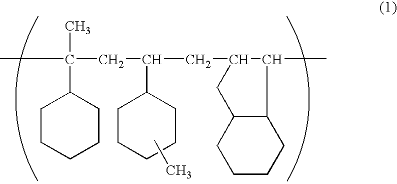

(4) The adhesive composition of any of the above-mentioned (1) to (3), wherein the above-mentioned rubber elastomer comprises a first rubber elastomer and a second rubber elastomer, and the first rubber elastomer and the second rubber elastomer are each independently at least one kind selected from polyisobutylene, polyisoprene, a butyl rubber and a styrene thermoplastic elastomer.
(5) The adhesive composition of the above-mentioned (4), wherein the above-mentioned first rubber elastomer has a weight average molecular weight of 1,600,000-5,400,000, and the above-mentioned second rubber elastomer has a weight average molecular weight of 36,000-75,000.
(6) A patch comprising a support and an adhesive layer comprising the adhesive composition of any of the above-mentioned (1) to (5), which is provided on at least one surface of the support.
(7) A patch preparation comprising the patch of the above-mentioned (6), wherein the patch has an adhesive layer comprising a percutaneously absorbable drug (excluding bisoprolol).

The adhesive composition for patch of the present invention contains a tackifier having a weight average molecular weight of 1200-2500. As a result, the composition can suppress a decrease in the skin adhesion while maintaining the cohesive force of the rubber elastomer. Therefore, by combining the above-mentioned tackifier with a rubber elastomer having a comparatively high molecular weight, still higher cohesive force can be realized, and an adhesive composition for patch that resists easy decrease in the skin adhesion can be obtained. Accordingly, the present invention provides an adhesive composition for patch, which can simultaneously achieve a high cohesive force and a high level of adhesion to the skin.

When an adhesive composition contains a large amount of an organic liquid component, the cohesive force of the adhesive composition tends to decrease. However, when the adhesive composition contains a tackifier having a weight average molecular weight of 1200-2500 according to the present invention, the skin adhesion of the adhesive composition does not decrease easily even when a rubber elastomer having a comparatively high molecular weight and high cohesive force is used for the adhesive composition. That is, the adhesive composition for patch of the present invention can contain a large amount of an organic liquid component, and even in such case, an adhesive composition having good adhesive property and capable of simultaneously achieving a high cohesive force and high skin adhesion can be obtained. Consequently, the adhesive composition of the present invention can contain a large amount of an organic liquid component, which in turn minimizes the irritation to the skin on detachment.

In this way, the adhesive composition for patch of the present invention can afford the aforementioned good adhesive property. Therefore, when the composition is used for the adhesive layer of a patch, the patch exhibits high adhesiveness to the skin, reduces an adhesive residue on the skin surface upon detachment therefrom, suppresses an adhesive residue on a release film from the adhesive layer and delamination of a release film during production or use, and enables re-adhesion to the skin.

Furthermore, the patch preparation of the present invention containing a percutaneously absorbable drug in the adhesive layer shows high transdermal absorbability of the drug and enables dissolution and retention of a large amount of the drug in the adhesive layer, whereby a treatment effect can be increased.

### BRIEF DESCRIPTION OF THE DRAWING

FIG. 1 is a sectional view showing one embodiment of the patch of the present invention, wherein 1 is a support, 2 is an adhesive layer, 3 is a release film, and 10 is a patch.

### DETAILED DESCRIPTION OF THE INVENTION

The present invention is explained in detail in the following by referring to a preferable embodiment. In the explanation of the drawing, the same symbol shows the same element, and overlapping explanations are omitted. For convenience, moreover, the size ratios in the drawing do not always match the corresponding ratios in the explanation.

### (Adhesive Composition for Patch)

Firstly, the adhesive composition for patch of the present invention is explained.

The adhesive composition for patch of the present invention (hereinafter sometimes to be simply referred to as “an adhesive composition”) comprises a rubber elastomer and a tackifier having a weight average molecular weight of 1200-2500.

The rubber elastomer is not particularly limited as long as it can be used as an adhesive for a patch. From the aspects of price, handlability and the like, polyisobutylene, polyisoprene, butyl rubber and styrene thermoplastic elastomer (e.g., styrene-diene-styrene block copolymer such as styrene-isoprene-styrene block copolymer etc. and the like) are preferable, and polyisobutylene is more preferable. These can be used alone or in a mixture of two or more kinds.

The molecular weight of the rubber elastomer is not particularly limited as long as the adhesive satisfies adhesiveness and cohesive force required of an adhesive. To achieve higher cohesive force, a first rubber elastomer having a comparatively high molecular weight is preferably used. To achieve more superior skin adhesion, a second rubber elastomer having a comparatively low molecular weight is preferably used. To simultaneously achieve these properties, a mixture of rubber elastomers including at least these two kinds is preferably used.

The weight average molecular weight (Mw) of the first rubber elastomer is preferably 1,600,000-5,400,000, more preferably 1,800,000-4,800,000, most preferably 3,000,000-4,500,000. When Mw of an adhesive is less than 1,600,000, the adhesive tends to have difficulty in achieving an internal cohesive force. On the other hand, when Mw exceeds 5,400,000, the adhesive tends to show degraded skin adhesion and lower tackiness.

The molecular weight (Mw) of the second rubber elastomer is preferably 36,000-75,000, more preferably 40,000-60,000, most preferably 45,000-55,000. When Mw is less than 36,000, a sticky adhesive is obtained, and the stickiness may stain the skin surface during and after application. On the other hand, when Mw exceeds 75,000, the skin adhesion and tackiness of the adhesive tend to decrease. Here, Mw means Mw based on polystyrene as measured by a gel penetration chromatography (GPC) method. The measurement conditions are as follows.

eluent: tetrahydrofuran (THF),

sample concentration (THF solution): 0.1% (W/V),

column: G4000H_{XL}+G2000H_{XL}+G1000H_{XL} (manufactured by Tosoh Corporation, each 7.8 mmφ×300 cm),

column temperature: 40° C.,

detector: differential refractometer (RI)

The amount of a rubber elastomer to be blended is preferably 15-60 wt %, more preferably 15-55 wt %, relative to the total weight of the adhesive composition. When the total amount of the rubber elastomer is less than 15 wt %, internal cohesive force necessary for the adhesive composition may be difficult to obtain. On the other hand, when it exceeds 60 wt %, the skin adhesion and tackiness of the adhesive composition may be degraded. Even when the adhesive composition consists of two or more kinds of rubber elastomers having different molecular weights, the total amount of rubber elastomer is similar to the above. In the present invention, the high cohesive force and a high level of adhesion to the skin can be simultaneously achieved even when the amount of a rubber elastomer to be used is less than the amount generally used in the art (e.g., not more than about 30 wt %, preferably not more than 27 wt %).

The blending ratio (weight ratio) of the first rubber elastomer and the second rubber elastomer (former:latter) is preferably 1:0.1-1:3, more preferably 1:0.1-2.5. When the ratio is less than 1:0.1, the skin adhesion may become insufficient, and when it exceeds 1:3, the cohesive force may become insufficient.

As mentioned above, the rubber elastomer may be a mixture of two or more kinds. When such mixture is employed, the respective elastomers may be the same or different. From the aspect of compatibility of the two, they are preferably of the same kind.

As the tackifier to be used in the present invention, those known in the art can be appropriately selected and used as long as Mw thereof is 1200-2500. For example, petroleum resins (e.g., liquid paraffin, alicyclic saturated hydrocarbon resin), terpene resin, rosin resin, coumarone-indene resin, styrene resins (e.g., styrene resin, a-methylstyrene) and the like are preferable. Of these, since good tackiness is obtained, petroleum resins, particularly alicyclic saturated hydrocarbon resins, are more preferable. As the tackifier, one kind alone or a combination of two or more kinds can be used.

The alicyclic saturated hydrocarbon resin is not particularly limited. However, since good skin adhesion and good tackiness are afforded, a hydrogenated resin obtained by thermally decomposing naphtha, polymerizing the obtained unsaturated hydrocarbon to give a thermoplastic resin, and adding hydrogen to form an alicyclic structure is preferably used. Particularly, an alicyclic saturated hydrocarbon resin having a repeat unit represented by the following formula (1) is particularly preferable:

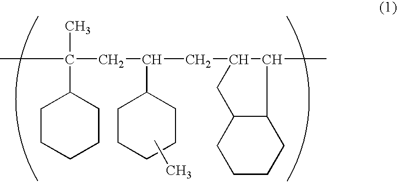

(hereinafter to be referred to as “resin (1)”).

To afford good tackiness, the tackifier needs to have Mw of 1200-2500, preferably 1300-2500, more preferably 1500-2400, most preferably 1700-2300. When Mw of the tackifier is less than 1200, stickiness may be developed which may stain the skin surface during and after application. On the other hand, when Mw exceeds 2500, an effect of a tackifier is difficult to show and the tackiness tends to decrease. Here, Mw is also based on polystyrene measured by a gel penetration chromatography (GPC) method where the measurement conditions are as mentioned above.

When the aforementioned first rubber elastomer is used to impart high cohesive force to an adhesive composition, a tackifier needs to be added since the composition itself does not have skin tackiness. According to the finding of the present inventors, a tackifier having Mw of about 1100 fails to impart skin tackiness. It was predicted that, in such a case, a tackifier having a lower molecular weight than as indicated above may be added to afford stronger tackiness. Despite such prediction, it has been unexpectedly found that desired skin tackiness can be attained only by adding a tackifier having a higher molecular weight than as indicated above. While the mechanism of this phenomenon is not necessarily clear, the present inventors assume that the first rubber elastomer having a comparatively high molecular weight contains a comparatively large space between molecules due to its long polymer chains, and a tackifier having a comparatively high molecular weight fills the space, whereby the skin tackiness of the adhesive composition is efficiently exhibited.

The amount of the tackifier to be blended is preferably 15-55 wt %, more preferably 20-50 wt %, most preferably 25-50 wt %, relative to the total weight of the adhesive composition. When the amount of the tackifier is less than 15 wt %, the tackiness may be poor, whereas when it exceeds 55 wt %, the cohesive force in the adhesive may decrease markedly, showing a tendency toward destruction.

The adhesive composition of the present invention can contain an organic liquid component. As the organic liquid component for an adhesive composition containing a drug, a liquid compound having a function to enhance solubility and diffusion of the drug and capable of improving the transdermal absorption of the drug is preferably used. Specific examples of such transdermal absorption promoter include the following compounds. Examples of the compound mainly enhancing drug solubility include glycols such as ethylene glycol, diethylene glycol, propylene glycol, triethylene glycol, polyethylene glycol, polypropylene glycol and the like, and examples of the compound mainly enhancing drug diffusion include fats and oils such as olive oil, castor oil, squalane, lanolin etc. and the like. Examples of others include hydrocarbons such as liquid paraffin and the like; various surfactants; ethoxylated stearyl alcohol; glycerol monoesters such as oleic monoglyceride, caprylic monoglyceride, lauryl monoglyceride and the like; glycerol diester, glycerol triester or a mixture thereof; higher fatty acid esters such as ethyl laurate, isopropyl myristate, isotridecyl myristate, octyl palmitate, isopropyl palmitate, ethyl oleate, diisopropyl adipate and the like; higher fatty acids such as oleic acid, caprylic acid and the like; N-methylpyrrolidone; 1,3-butanediol and the like.

The amount of the organic liquid component to be blended is preferably 20-50 wt %, more preferably 30-40 wt %, relative to the total weight of the adhesive composition. When the amount is less than 20 wt %, the skin irritation tends to intensify. On the other hand, when the amount exceeds 50 wt %, cold flow may not be suppressed sufficiently, cohesive force decreases markedly and cohesive failure tends to occur easily.

Furthermore, the adhesive composition of the present invention may contain regular additives, for example, UV absorber, light stabilizer, release adjuster, plasticizer, softener, filler, colorant (pigment, dye etc.), anti-aging agent, surfactant and the like. The amount of such component to be blended can be appropriately determined according to the object of use, the kind and composition of the adhesive composition and the like.

### (Patch)

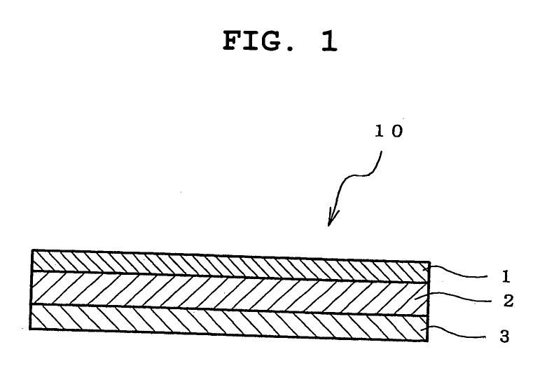

FIG. 1 is a sectional view showing one preferable embodiment of the patch of the present invention.

Patch 10 is provided with a support 1 and an adhesive is layer 2 laminated on one surface of the support 1, and a release film 3 laminated on the adhesive layer 2. The adhesive layer 2 characteristically comprises the aforementioned adhesive composition for patch.

The support is not particularly limited and, in view of the handling property, flexibility and anchor property, a single film of polyester, polyamide (e.g., nylon (trademark)), polyvinylidene chloride (e.g., saran (trademark)), polyethylene, polypropylene, polyvinyl chloride, ethylene-ethyl acrylate copolymer, polytetrafluoroethylene, ionomer resin (e.g., Surlyn (trademark)), metal foil and the like, a laminate film thereof and the like can be used. To improve anchor property, a support comprised of a single layer film made of the above-mentioned material and a porous film laminated thereon is more preferable. In this case, an adhesive layer is desirably laminated on the porous film side. While the thickness of the support is not particularly limited, it is preferably 10-200 μm, more preferably 10-100 μm.

To improve the anchor property between the support and the adhesive layer, a non-woven fabric or a woven fabric may be laminated on the support. The material of the non-woven fabric and the woven fabric is not particularly limited, and the material can be appropriately selected from those generally used in the field of patch. Examples of such material include polyester, polypropylene, polyamide and the like. The basis weight of the non-woven fabric or woven fabric is generally 1-100 g/m², preferably 6-50 g/m². It is particularly preferably 6-30 g/m², since flexibility and a feeling of close adhesion to the skin surface are fine during application.

In addition, the patch of the present invention preferably contains a release film laminated on the adhesive layer to protect the adhesive surface before use. The release film is not particularly limited, and a known release film can be used. For example, films of polyester, polyvinyl chloride, polyvinylidene chloride, poly(ethylene terephthalate) and the like, papers such as quality paper, glassine paper and the like, or a laminate film of quality paper, glassine paper etc. and polyolefin, and the like after a detach treatment by applying silicone resin, fluorine resin and the like to the surface to be in contact with the adhesive layer can be used. The thickness of the release sheet is generally 10-200 μm, preferably 25-100 μm.

In addition, the release force of a release film from the adhesive layer is preferably 10-400 mN/cm. When the release force is less than 10 mN/cm, problems tend to occur such as delamination of a patch from a release film when taking out the patch from a bag and rolling up of a patch during production. On the other hand, when the force exceeds 400 mN/cm, an adhesive composition containing a drug remains on the release film, which decreases the drug content and treatment effect. A further preferable range of the release force is 20-300 mN/cm.

The production method of the patch of the present invention is not particularly limited and, for example, it can be produced by dissolving the above-mentioned adhesive composition in a suitable solvent such as toluene, hexane and the like, applying the obtained solution on a support, and drying the solution. It is also possible to produce the patch by applying the above-mentioned solution onto a release film, drying the solution to form an adhesive layer on the release film, and adhering a support to the adhesive layer. The thickness of the adhesive layer is preferably 20-300 μm, more preferably 50-250 μm, from the aspects of retention of cohesive force and prevention of adhesive residue. The shape of the patch is not particularly limited and, for example, it may be a tape or sheet.

The thus-obtained patch shows high adhesion to the skin, can detach from the skin surface without adhesive residue and skin irritation, and permits re-adhesion. Therefore, it is useful as a first-aid adhesive plaster, a roll of long adhesive plaster, a large size adhesive plaster with a pad, a dressing and the like.

### (Patch Preparation)

The patch preparation of the present invention has a laminate structure similar to that of the above-mentioned patch 10, but differs therefrom in that it contains a percutaneously absorbable drug (excluding bisoprolol) in an adhesive layer 2. Since the patch preparation of the present invention contains a percutaneously absorbable drug in the adhesive layer, it can be used as a transdermally absorbable preparation.

The transdermally absorbable drug is not particularly limited as long as it can be administered through the skin of mammals such as human and the like, i.e., transdermal absorbability. Specific examples of the drug include general anesthetics, hypnotic sedatives, antiepileptic drugs, antipyretic analgesic antiphlogistic drugs, anti-vertigenous drugs, psychoneurotic drugs, topical anesthetics, skeleton muscle relaxants, autonomic drugs, antispasmodic drugs, anti-parkinsonian drugs, anti-histamine drugs, cardiac stimulants, drugs for arrhythmia, diuretics, hypotensive drugs, vasoconstrictors, colonary vasodilators, peripheral vasodilators, arteriosclerosis drugs, drugs for circulatory organ, anapnoics, antitussive expectorants, hormone drugs, external drugs for mattery diseases, nalgesic-antipruritic-styptic-antiphogistic drugs, drugs for parasitic dermatic diseases, drugs for arrest of bleeding, gout treatment drugs, drugs for diabetes, drugs for anti-malignant tumor, antibiotics, chemical therapy drugs, narcotics, anti-schizophrenia drugs, antidepressants, quit smoking aids and the like.

In addition, when the transdermally absorbable drug is a solid drug, it may crystallize in the adhesive layer. However, since the present invention can advantageously suppress the crystallization, solid drugs can be preferably used as the transdermally absorbable drug. Here, the solid drug means a drug which is solid at room temperature (25° C.), in other words, a drug having a melting point of not less than 25° C.

When the transdermally absorbable drug is a liquid drug, moreover, it may bleed out from an adhesive layer. However, since the present invention can advantageously suppress the bleeding out, liquid drugs are also preferable as the transdermally absorbable drug. Here, the liquid drug means a drug which is liquid at room temperature (25° C.), in other words, a drug having a melting point of less than 25° C. Here, the melting point means a value measured by DSC (model number DSC6220, manufactured by Seiko Instruments Inc. (SII)) according to JIS K7121.

The amount of the transdermally absorbable drug to be added is appropriately determined according to the kind of drug, administration object and the like. Generally, a drug is preferably contained in a proportion of 0.5-40 wt %, more preferably 1-30 wt %, in an adhesive layer. When the amount is less than 0.5 wt %, absorption of the drug in an amount sufficient to provide a pharmacological effect is difficult. On the other hand, when the amount exceeds 40 wt %, skin adhesion tends to decrease.

The patch preparation of the present invention can also be produced by adding a percutaneously absorbable drug to the above-mentioned adhesive composition, dissolving the composition in a solvent, and processing the solution in the same manner as in the aforementioned patch.

The patch preparation of the present invention is adhered to the skin surface once in about 1 or 2 days, though subject to change depending on the kind of drug, age, body weight and symptom of patient, and the like.

### EXAMPLES

The present invention is explained in detail in the following by referring to Examples, which are not to be construed as limitative. The materials used in the Examples and the like were as follows.

polyisobutylene A: Mw 4,000,000

polyisobutylene B: Mw 51,000

tackifier T1: alicyclic saturated hydrocarbon resin [resin(1)], Mw 2,140

tackifier T2: alicyclic saturated hydrocarbon resin [resin(1)], Mw 1,530

tackifier T3: alicyclic saturated hydrocarbon resin [resin(1)], Mw 1,130

organic liquid component: isopropyl myristate (IPM)

### Examples 1-5 and Comparative Examples 1-2

A viscous toluene solution of an adhesive composition for patch having the composition ratios described in Table 1 was prepared, and the obtained solution was applied onto a poly(ethylene terephthalate) (PET) release film, which had undergone a silicone detach treatment, such that the thickness after drying was 80 μm, and dried at 100° C. for 5 min in a hot-air circulation oven to give an adhesive layer. The adhesive layer was adhered to a non-woven fabric side of a laminate film consisting of a PET film (thickness 2 μm) and a PET non-woven fabric (12 g/m²) to give a sheet-like patch. The amount of each component described in Table 1 is the ratio (wt %) relative to the total weight of the adhesive composition.

TABLE 1

| | |
|---|---|
| polyisobutylene | organic liquid |

| | first | second | tackifier | component |
|---|---|---|---|---|
| Example 1 | A 16.8 | B 18.2 | T1 35.0 | IPM 30.0 |
| Example 2 | A 16.8 | B 18.2 | T2 35.0 | IPM 30.0 |
| Example 3 | A 22.4 | B 26.6 | T1 21.0 | IPM 30.0 |
| Example 4 | A 9.8 | B 11.2 | T1 49.0 | IPM 30.0 |
| Example 5 | A 12.0 | B 13.0 | T1 35.0 | IPM 40.0 |
| Comparative | A 16.8 | B 18.2 | T3 35.0 | IPM 30.0 |
| Example 1 | | | | |
| Comparative | A 12.0 | B 13.0 | T3 35.0 | IPM 40.0 |
| Example 2 | | | | |

The patches produced in Examples and Comparative Examples were subjected to the following tests, the results of which are shown in Table 2.

### <Skin Adhesion>

The patches prepared in the Examples and Comparative Examples were adhered to the antebrachial region of volunteers and the adhesion state after 24 hr was visually evaluated. The evaluation criteria are as follows.

◯: no delamination
Δ: end delamination was observed
x: detachment was observed

### <Adhesive Residue on the Skin>

After the completion of the above-mentioned skin adhesion test, the patch was detached from the antebrachial region. The state of the adhesive layer and skin surface was visually observed, and the presence of adhesive residue from the adhesive layer on the skin was evaluated. The evaluation criteria were as follows.

◯: No adhesive residue on skin surface upon detachment
Δ: Slight adhesive residue on skin surface upon detachment
x: Presence of adhesive residue on skin surface upon detachment

### <Re-Adhesion>

After the completion of above-mentioned skin adhesion test, the patch detached from the antebrachial region was adhered again to the antebrachial region, and adhesion capability was evaluated. The evaluation criteria were as follows.

◯: re-adherable
Δ: re-adherable but considerably weak skin adhesion
x: re-adhesion not possible

### <Adhesive Residue on Release Film>

The release film was peeled off from the patches produced in the Examples and Comparative Examples, and the amount of the adhesive attached to the release film was visually observed to evaluate adhesive residue on the release film from the adhesive layer. The evaluation criteria were as follows.

◯: Adhesive was not confirmed visually
Δ: Presence of slight attachment
x: Clear presence of attachment

### <Delamination of Release Film>

The patches prepared in the Examples and Comparative Examples were bent with the support side facing inside, and is the interlayer strength between the release film and the adhesive layer was visually observed. The evaluation criteria were as follows.

◯: No delamination of release film and adhesive layer
Δ: Slight delamination of release film and adhesive layer
x: Clear delamination of release film and adhesive layer

TABLE 2

| | | | | adhesive | delamination |
|---|---|---|---|---|---|
| | skin | adhesive | | residue on | of |
| | ad- | residue | re- | release | release |
| | hesion | on skin | adhesion | film | film |
| Example 1 | ◯ | ◯ | ◯ | ◯ | ◯ |
| Example 2 | Δ | ◯ | ◯ | ◯ | ◯ |
| Example 3 | ◯ | ◯ | ◯ | ◯ | ◯ |
| Example 4 | ◯ | ◯ | ◯ | ◯ | ◯ |
| Example 5 | ◯ | ◯ | ◯ | ◯ | ◯ |
| Comparative | X | ◯ | ◯ | ◯ | X |
| Example 1 | | | | | |
| Comparative | X | Δ | Δ | Δ | X |
| Example 2 | | | | | |

### Examples 6-10 and Comparative Examples 3-4

In the same manner as in the above-mentioned Examples except that a transdermally absorbable adhesive composition was prepared by adding 1 part by weight of indomethacin (anti-inflammatory agent, solid drug) to 100 parts by weight of the components described in Table 1, a patch preparation was obtained.

As is clear from Table 2, the patch of Example 2 showed somewhat lower skin adhesion performance, but the patches of Examples 1 and 3-5 showed good skin adhesion. That is, it was found that T1 and T2, particularly T1, were superior in skin adhesion. In addition, the patches of the Examples were confirmed to be superior in the following aspects as compared to the patches of Comparative Examples.

(i) high adhesion to skin,
(ii) no adhesive residue on skin surface on detachment from skin and low irritation to skin on detachment,
(iii) no adhesive residue on release film from adhesive layer during production and use, and suppressed delamination of release film, and
(iv) possible re-adhesion to skin.

In addition, it was confirmed that the patch preparations of Examples 6-10 also have the properties of the above-mentioned (i)-(iv), and provide an antiphlogistic and analgesic effect. In contrast, the patch preparations of Comparative Examples 3-4 showed results similar to those of Comparative Examples 1-2, and they were confirmed to be inferior in the properties to the patch preparations of Examples 6-10.

Therefore, the patches and patch preparations of the Examples were confirmed to have simultaneously afforded high cohesive force and skin adhesion at high levels.

1. An adhesive composition for patch, comprising a rubber elastomer and a tackifier having a weight average molecular weight of 1200-2500.

2. The adhesive composition of claim 1, wherein said tackifier is at least one kind selected from a petroleum resin, a terpene resin, a rosin resin, a coumarone-indene resin and a styrene resin.

3. The adhesive composition of claim 2, wherein the above-mentioned petroleum resin is a saturated alicyclic hydrocarbon resin having a repeat unit represented by the following formula (1):

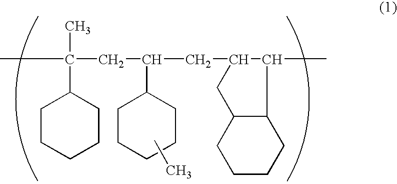

4. The adhesive composition of claim 1, wherein said rubber elastomer comprises a first rubber elastomer and a second rubber elastomer, and the first rubber elastomer and the second rubber elastomer are each independently at least one kind selected from polyisobutylene, polyisoprene, a butyl rubber and a styrene thermoplastic elastomer.

5. The adhesive composition of claim 4, wherein said first rubber elastomer has a weight average molecular weight of 1,600,000-5,400,000, and said second rubber elastomer has a weight average molecular weight of 36,000-75,000.

6. A patch comprising a support and an adhesive layer comprising the adhesive composition of claim 1, which is provided on at least one surface of the support.

7. A patch preparation comprising the patch of claim 6, wherein the patch has an adhesive layer comprising a percutaneously absorbable drug excluding bisoprolol.

8. A patch comprising a support and an adhesive layer comprising the adhesive composition of claim 2, which is provided on at least one surface of the support.

9. A patch preparation comprising the patch of claim 8, wherein the patch has an adhesive layer comprising a percutaneously absorbable drug excluding bisoprolol.

10. A patch comprising a support and an adhesive layer comprising the adhesive composition of claim 3, which is provided on at least one surface of the support.

11. A patch preparation comprising the patch of claim 10, wherein the patch has an adhesive layer comprising a percutaneously absorbable drug excluding bisoprolol.

12. A patch comprising a support and an adhesive layer comprising the adhesive composition of claim 4, which is provided on at least one surface of the support.

13. A patch preparation comprising the patch of claim 12, wherein the patch has an adhesive layer comprising a percutaneously absorbable drug excluding bisoprolol.

14. A patch comprising a support and an adhesive layer comprising the adhesive composition of claim 5, which is provided on at least one surface of the support.

15. A patch preparation comprising the patch of claim 14, wherein the patch has an adhesive layer comprising a percutaneously absorbable drug excluding bisoprolol.
